(19) United States
(12) Patent Application Publication
(10) Pub. No.: US 2008/0000486 A1
(43) Pub. Date: Jan. 3, 2008

(54) STRAPS FOR RESTRAINING A PATIENT'S ARMS

(75) Inventor: Michael Wilson, Winnsboro, TX (US)

Correspondence Address: CARSTENS & CAHOON, LLP, P O BOX 802334, DALLAS, TX, 75380, US

(73) Assignee: Itec Manufacturing, Ltd., Winnsboro, TX (US)

(21) Appl. No.: 11/424,125

(22) Filed: Jun. 14, 2006

(51) Int. Cl. A61B 19/00

(52) U.S. Cl. 128/898

(57) ABSTRACT

An arm restraint device and method for use by EMS workers to restrain the arms of a patient lying on a backboard, gurney, or similar immobilizing transport device. A strap having two outer loops for encircling each arm is joined by centrally located buckles. Each loop is placed over a patient's arms and positioned slightly above the elbow. A single pull of the strap between the buckles defining the two outer loops adjusts the outer loops to secure the patient's arms. The patient's arms are held parallel to the patient's sides but remain accessible to EMS workers even while restrained. The restraint device functions independently of the other straps on the immobilizing transport device to allow repositioning of the restraint without loosening the other straps and risking unexpected movement of the patient.

### CROSS-REFERENCES TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to a patient arm restraint. More specifically, the present invention relates to an arm restraint apparatus and method for its use to safely and efficiently restrain the arms of a patient lying on a stretcher or backboard device.

2. Description of Related Art

When responding to a medical emergency, it is quite common for an EMS worker to encounter a patient in need of transport. Often, the patient is unable to move or assist in moving on their own due to a lack of consciousness. When the EMS worker places the patient on a backboard or gurney, the patient's arms tend to drop from the backboard or gurney and dangle freely. In this state, the patient's freely moving arms can be further injured and/or cause the EMS worker great difficulty in stabilizing and moving the patient. For example, if the patient requires administration of a peripheral IV, the IV might easily dislodge and fail should the patient's arm drop unexpectedly from the backboard. Also, the patient could suffer direct injury to an arm or hand, or even compression injuries to an elbow or shoulder should the patient's arms drop unexpectedly while placing the patient into an ambulance.

Most backboards and gurneys have attached straps that are used to restrain a patient by wrapping firmly around the patient's torso and legs. While this works fine for overall immobilization, it does not work well for the patient's arms. FIG. 1 illustrates the problem with transporting a patient with unrestrained arms on a backboard. For instance, if the torso straps are utilized in an effort to restrain the arms by trapping the arms next to the patient's torso, the arms are not readily accessible for administration of an IV or taking of vital signs. Also, should the arm need to be moved for any reason, the torso strap will have to be loosened. Loosening a torso strap, especially while transporting a patient, greatly increases the chances of the patient moving or shifting on the backboard or gurney unexpectedly. This could result in increased injuries to the patient and quite possibly the EMS worker. Finally, the tension placed on the torso straps to sufficiently immobilize a patient may be high enough to reduce or even interrupt blood circulation in the arms, resulting in further injury.

Time is of the essence in an emergency rescue situation. The more rapidly a patient can be immobilized, stabilized, and transported to a medical facility, the greater the odds are for full recovery. Thus, when securing a patient to a backboard or gurney, it is vital that the arm restraint straps be easy to apply. While it may seem as though restraining a patient's arms by trapping them beneath the torso restraints may be the quickest method, it actually increases the time required to tend to the patient. For example, if an arm is needed to administer an IV or medications, the arm will need to be freed from the restraint. Also, stopping the ambulance so as to reposition the patient within the torso straps will further increase the transport delay.

Therefore, a need exists for a device that can restrain a patient's arms when the patient is unconscious and unable to assist. Further, a need exists for an arm restraint device that can restrain a patient's arms in order to assist EMS workers in safely transporting the patient. Further, a need exists for an arm restraint device that is sturdy, simple to use, and easy to store. Further, a need exists for an arm restraint device that is independent of any straps used to immobilize a patient on a backboard, gurney, or similar transport device. Further, a need exists for an arm restraint device that allows an EMS worker to access the arms without affecting the tension on the straps used to immobilize the patient. Further, a need exists for an arm restraint device that maintains control of the arms while still allowing for the insertion of an IV. And further, a need exists for an arm restraint device that does not increase the risk of injury to the patient. The present invention fills these needs and other needs as detailed more fully below.

### BRIEF SUMMARY OF THE INVENTION

The present invention is directed to an arm restraint apparatus and method for its use. The arm restraint apparatus in its preferred embodiment is intended for use by EMS workers or others who are involved in patient immobilization and transport. It serves to restrain the arms of a patient when lying on a backboard, gurney, or other immobilizing transport device. After the patient is securely immobilized on the transport device, the restraint device is slipped gently over each of the patient's arms. Once in place, a simple pull on the restraint device's adjustment strap is all that is necessary to safely and effectively restrain the patient's arms against the patient's torso. Though restrained, the device still allows access to the arms by EMS workers for maintenance of an IV or administration of other medical procedures.

Accordingly, it is one general object of the invention to provide an arm restraint device that is inexpensive, simple to operate, and quick to install and adjust.

It is another general object of the invention to provide an arm restraint device that functions independently of other straps used to immobilize the patient on a backboard, gurney, or other transport device.

The invention accordingly comprises the features described more fully below. The scope of the invention will be indicated in the claims. Further objects of the present invention will become apparent in the following detailed description when read in light of the drawings.

### BRIEF DESCRIPTION OF THE SEVERAL VIEWS OF THE DRAWING(S)

The present invention will be more fully understood by reference to the following detailed description of the preferred embodiments of the present invention when read in conjunction with the accompanying drawings, in which like reference numerals refer to like parts throughout the views, wherein:

FIG. 1 is an illustration of a patient on a backboard being transported with unrestrained arms.

FIG. 2 is an illustration of an embodiment of the present invention.

FIG. 3 is an illustration of the embodiment of the present invention in use on a patient. More specifically, this is a depiction of the first step wherein the apparatus is slipped over the arms of a patient secured to a backboard.

FIG. 4 is an illustration of the embodiment of the present invention in use on a patient. More specifically, this is a depiction of the second step wherein the apparatus is positioned on the arms immediately above the elbows.

FIG. 5 is an illustration of the embodiment of the present invention in use on a patient. More specifically, this is a depiction of the third step wherein the center tensioning strap is adjusted to remove slack in the loops of the apparatus.

FIG. 6 is an illustration of the embodiment of the present invention in use on a patient. More specifically, this is a depiction of the fourth step wherein the center tensioning strap is further adjusted to pull the patient's arms in securely to the patient's sides.

FIG. 7 is an illustration of the embodiment of the present invention in use on a patient. More specifically, this is a depiction of the apparatus in use on a patient after it has been properly adjusted.

FIG. 8 is an illustration of the embodiment of the present invention in use on a patient. More specifically, this is a depiction of the patient on a backboard being transported with the arms restrained using an embodiment of the present invention.

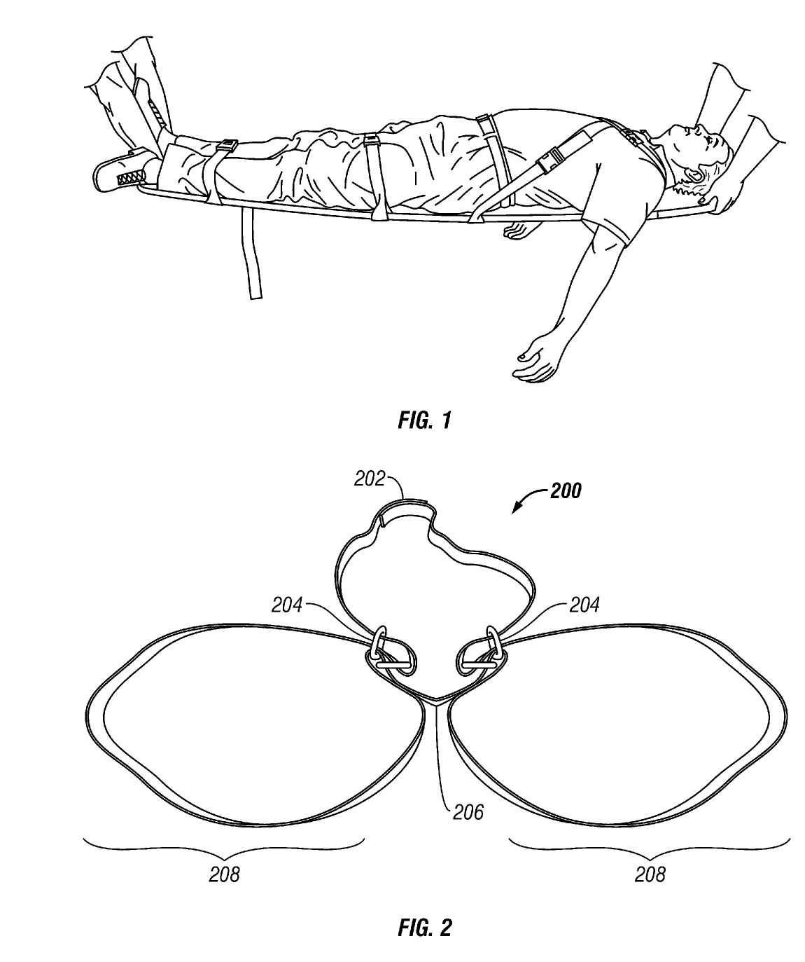

Where used in the various figures of the drawing, the same reference numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### REFERENCE NUMERALS

200 arm restraint
202 tensioning strap
204 buckle
206 center
208 loop

### DETAILED DESCRIPTION OF THE INVENTION

Referring now to the provided drawings, wherein similar reference numbers represent the equivalent component throughout the several views, FIG. 2 illustrates an embodiment of the present invention configured as an arm restraint 200 device.

In this embodiment, the arm restraint 200 is made from a single length of nylon strap. However, a person having ordinary skill in the art of strap making would understand that multiple sections may also be combined to create the desired length. Other materials such as polypropylene, polyester, leather or canvas strap may also be used for the device. The length chosen for the arm restraint 200 strap should be sufficient to allow for the formation of two loops 208, one for each arm. The length should allow for enough adjustability of the arm restraint 200 to allow it to slip easily over both arms of any sized patient. In this embodiment the strap length is approximately 64 inches. This affords enough adjustment to allow utilization of the arm restraint 200 on adult and pediatric patients alike. Deviation from this length is acceptable because once properly adjusted, the size of the tensioning strap 202 portion will compensate for any excess length.

As illustrated in FIG. 2, the current embodiment features four D-rings configured as adjustable buckles 204. Each loop 208 utilizes two D-rings in a double D-ring configuration that allows the tensioning strap 202 to move freely in one direction so as to increase tension in the outer loops 208 while at the same time maintaining the increased tension. To release the tension on the outer loops 208, the buckles 204 can be lifted away from the loop 208 strap, allowing the tensioning strap 202 to retract and the loop 208 to increase in diameter. While the current embodiment features a double D-ring configuration, the same effect can be achieved through the use of cam buckles, rectangular rings, circular rings, sliders, glides, or ladderloc buckles. In addition, the buckles 204 can be either metal or plastic due to the relatively low tension experienced through the proper use of the arm restraint 200.

With reference to FIG. 2, the current embodiment is constructed by initially threading the single piece of strap through each D-ring. The D-rings are combined in pairs, with each pair placed approximately two inches from the center of the strap on opposite sides of the strap center. The loose, outer length of each side of the strap is then folded over the respective D-ring pair and fastened upon itself so as to trap the buckle 204 in place. The strap in this embodiment is sewn together. However, any means of webbing attachment known to the art of strap making can be utilized. The two free strap ends are then once again woven through their respective buckles 204 in such a manner that two loops 208 are created. When properly woven, the free end of the strap can be pulled easily through the respective buckle 204 causing the respective loop 208 to contract. When the free end is released, the tension remains on the loop 208 and can only be released by pulling upward on the buckle 204. Once properly woven through the buckles 204, the two previously free ends are then attached together, creating the tensioning strap 202.

The strap material between the adjustable buckles 204 is the tensioning strap 202. When the tensioning strap 202 is completely released the loops 208 are at their largest. By grasping and pulling upward on the tensioning strap 202 (away from the center 206), the loops 208 are thus made smaller. While tension remains on the loops 208, lifting the top edge of the buckle 204 causes the tension to release, the loops 208 to expand, and the tensioning strap 202 to contract.

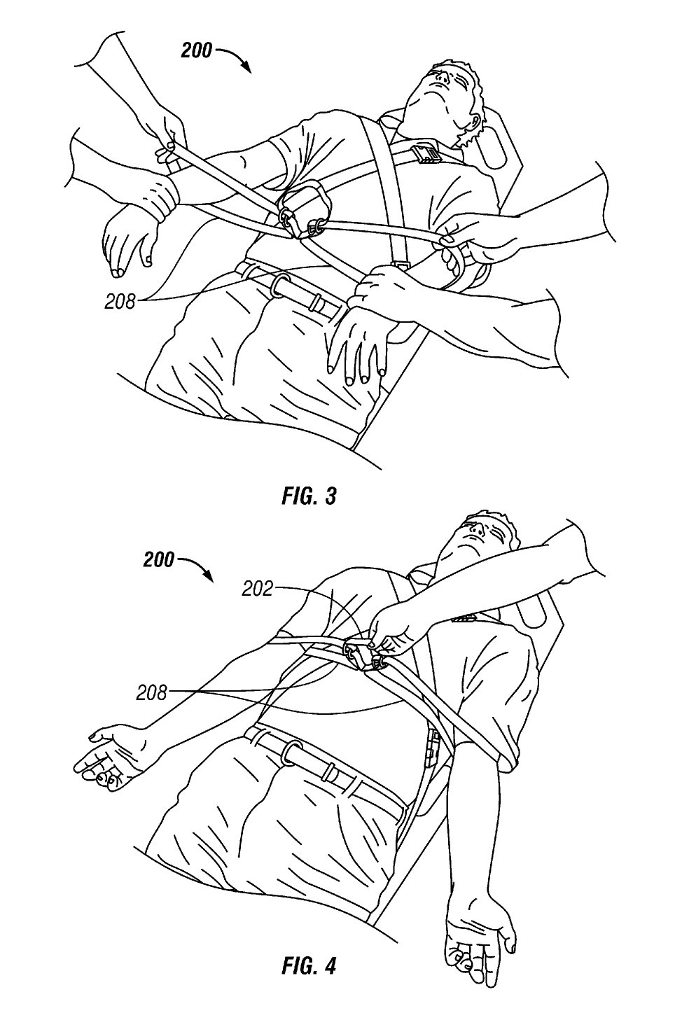

FIG. 3 through FIG. 8 illustrates the arm restraint 200 in the present embodiment in use on an unconscious patient. FIG. 3 shows the arm restraint 200 with the tensioning strap 202 completely released such that the loops 208 are fully extended. The arm restraint 200 is slipped over the arms of the patient, one loop 208 per arm. FIG. 4 shows proper placement of the loops 208 on the patient's arms, immediately above the elbows.

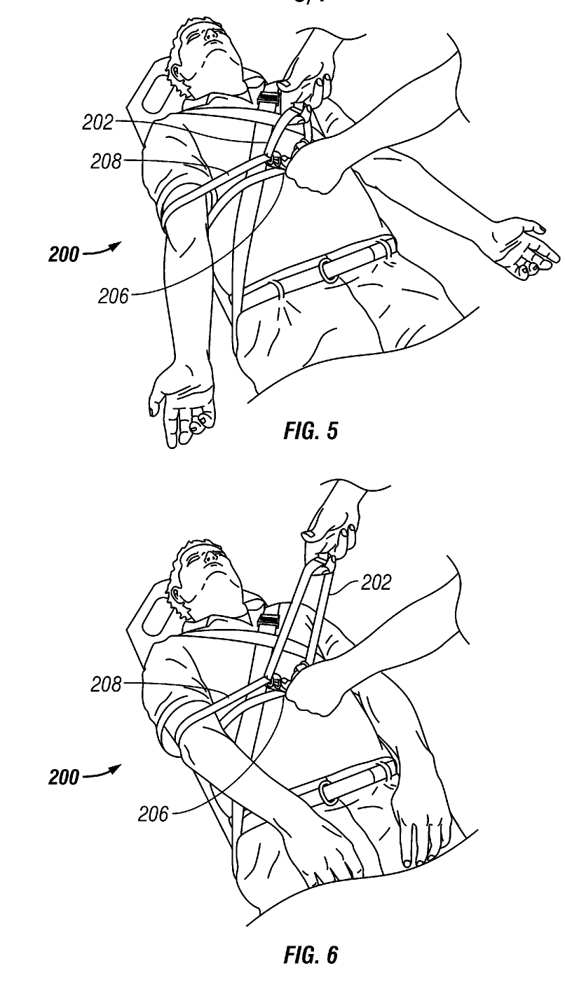

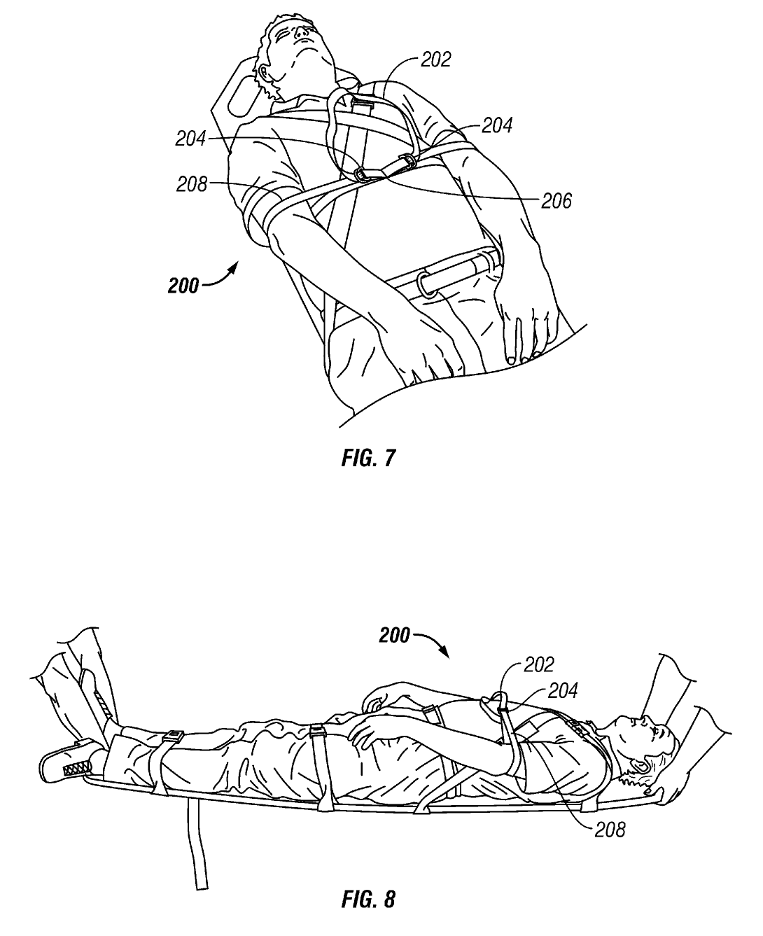

Once in position, the arm restraint can be adjusted. FIG. 5 illustrates the proper procedure for adjustment of the arm restraint 200. While holding the center 206, the tensioning strap 202 is pulled upward and away from the center 206. With reference to FIG. 6, as the tensioning strap 202 is pulled further upward the loops 208 retract and the patient's arms are pulled inward towards the abdomen. Because the loop 208 straps are pulled inward on the top side of the patient's arms, a rolling motion is created that turns the patient's palms inward. As shown in FIG. 7, once proper tension is established, the patient's arms will rest firmly and comfortably against the abdomen. The buckles 204 serve to retain the proper tension on the patient's arms when the grip on the tensioning strap 202 is released.

When transporting an unconscious patient with the arm restraint 200 device properly installed as shown in FIG. 8, the patient's arms are positively restrained in a comfortable position against the patient's abdomen. Because the arm restraint 200 works independently from the backboard straps used to immobilize the patient, the arm restraint can be adjusted or even removed without concern that the patient could suddenly shift or even fall from the backboard. This allows an EMS worker easy access to the patient's arms for medical monitoring or treatment (such as the administration of an IV). In addition, because the arm restraint 200 device is simple and efficient to use, a patient can be quickly and safely transported with the device installed.

This detailed description of the present invention has been presented for purposes of illustration and description, and is not intended to be exhaustive or limited to the invention in the form disclosed. Other modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

I claim:

1. A device for restraining the arms of a patient lying on a backboard, gurney, or similar patient immobilization and transportation means, said device comprising:
a strap, said strap having two outer loops for wrapping around and restraining each of said patient's arms and a tensioning portion for establishing the size of each of said two outer loops.

2. The device of claim 1 wherein said strap is chosen from the group consisting of nylon, polypropylene, polyester, leather and canvas.

3. The device of claim 1 wherein said strap is a single piece of material.

4. The device of claim 1 further comprising at least one buckle, said buckle for maintaining the size of said outer loops.

5. The device of claim 1 further comprising two buckles, each buckle for maintaining the size of one of said two outer loops.

6. The device of claim 1 wherein said tensioning portion is physically connected to both of said two outer loops, said tensioning portion capable of adjusting the size of both of said two outer loops simultaneously.

7. The device of claim 1 further comprising at least one buckle, said buckle chosen from the group consisting of D-ring, rectangular ring, circular ring, slider, glide, cam, and ladderloc buckles.

8. The device of claim 1 further comprising two buckles, said buckles chosen from the group consisting of D-ring, rectangular ring, circular ring, slider, glide, cam, and ladderloc buckles.

9. A method for using the device of claim 1, said method comprising the steps of:
a) securing the patient to said patient immobilization and transportation means;
b) sliding one loop of said device over each of the patient's arms;
c) positioning said loops immediately above the elbows of the patient's arms;
d) pulling on said tensioning portion of said device so as to rotate the patient's arms inward and pull the patient's arms toward the patient's abdomen; and
e) adjusting said tensioning portion of said device to maintain sufficient tension on said device such that the patient's arms are snuggly yet comfortably restrained against the patient's abdomen and thus prevented from dropping below said patient immobilization and transportation means.

10. A device for restraining the arms of a patient lying on a backboard, gurney, or similar patient immobilization and transportation means, said device comprising:
a strap, said strap having two outer loops for wrapping around and restraining said patient's arms and a tensioning portion for establishing the size of said two outer loops; and
at least one buckle, said at least one buckle for maintaining the size of said two outer loops.

11. The device of claim 10 wherein said strap is chosen from the group consisting of nylon, polypropylene, polyester, leather and canvas.

12. The device of claim 10 wherein said strap is a single piece of material.

13. The device of claim 10 wherein said buckle is either metal or polymer.

14. The device of claim 10 wherein said at least one buckle is chosen from the group consisting of D-ring, rectangular ring, circular ring, slider, glide, cam, and ladderloc buckles.

15. The device of claim 10 wherein said tensioning portion is physically connected to both of said two outer loops, said tensioning portion capable of adjusting the size of both of said two outer loops simultaneously.

16. A method for using the device of claim 10, said method comprising the steps of:
a) securing the patient to said patient immobilization and transportation means;
b) sliding one loop of said device over each of the patient's arms;
c) positioning said loops immediately above the elbows of the patient's arms;
d) pulling on said tensioning portion of said device so as to rotate the patient's arms inward and pull the patient's arms toward the patient's abdomen; and
e) adjusting said tensioning portion of said device to maintain sufficient tension on said device such that the patient's arms are snuggly yet comfortably restrained against the patient's abdomen and thus prevented from dropping below said patient immobilization and transportation means.
